(19) United States
(12) Patent Application Publication
(10) Pub. No.: US 2011/0022043 A1
(43) Pub. Date: Jan. 27, 2011

(54) DEVICE FOR THE TREATMENT OF SURFACES WITH A PLASMA GENERATED BY AN ELECTRODE OVER A SOLID DIELECTRIC VIA A DIELECTRICALLY IMPEDED GAS DISCHARGE

(76) Inventors: Dirk Wandke, Heiligenstadt (DE); Andy Kaemling, Birkenfelde (DE); Cindy Kaemling, Birkenfelde (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/666,866

(22) PCT Filed: Jun. 24, 2008
(86) PCT No.: PCT/EP08/05087

§ 371 Date: Aug. 27, 2010

(30) Foreign Application Priority Data
Jul. 3, 2007 (DE) 10-2007-030-915.7

(51) Int. Cl. A61B 18/14; H05H 1/24; A61N 5/00; A61L 2/18

(52) U.S. Cl. 606/41; 250/492.3; 422/28

(57) ABSTRACT

The invention relates to a device for treatment of surfaces with a plasma (2) generated by an electrode (1) over a solid dielectric (3) via a dielectrically impeded gas discharge, wherein the device has an effective surface (4) which is immediately adjacent to the plasma (2) during the treatment, wherein the form of the effective surface can be reversibly changed.

The invention relates to an apparatus for treatment of surfaces with a plasma which is produced by means of an electrode over a solid dielectric by a dielectric-barrier gas discharge.

In particular, DE 103 24 926 B3 discloses an apparatus for treatment of biological material containing living cells with a plasma produced by a gas discharge, in which an electrode is arranged at a distance from the biological material. A dielectric is arranged at a distance from the biological material, between the electrode and the biological material, and a high AC voltage is applied to the electrode in order to ignite the gas discharge, which has a dielectric barrier, between an active surface of the dielectric and the biological material. In this case, a solid dielectric is arranged in front of the electrode, without any separation, as a dielectric. One disadvantage in this case in that the biological materials generally have different topologies, as a result of which an irregular treatment for plasma that is produced takes place because of the inhomogeneous distances from the actual surface.

DE 101 27 035 A1 discloses a method for cleaning the air predominantly in living rooms and office areas using the silent electrical gas discharge, with ozone formation being virtually completely suppressed and with a dielectric for this purpose being arranged in a structured, applied, wire mesh composed of stainless steel.

DE 43 02 465 C1 discloses an apparatus for production of a dielectric-barrier discharge, having a gas-filled discharge area between two electrodes to which an ignition voltage can be applied and at least one of which is separated from the discharge area by a dielectric, with at least one electrode being a voltage-excited plasma in a gas, whose pressure is lower than the pressure in the discharge area.

U.S. Pat. No. 4,737,885 discloses a plasma generator having an excitation electrode and having an electrode arranged opposite it, in order to produce a plasma at high voltage, with a porous metal sheet being arranged between the electrodes and being provided with dielectric material with a metallic granular material.

EP 254 111 A1 discloses a high-power emitter, in particular for ultraviolet light, having a discharge area which is filled with filling gas and whose walls are formed on the one hand by a dielectric which is provided with first electrodes on its surface away from the discharge area, and on the other hand are formed from second electrodes or equally by a dielectric, which is provided with second electrodes on its surface facing away from the discharge area, having an AC power source, which is connected to the first and second electrodes, for feeding the discharge, and having means for guiding the radiation produced by the silent electrical discharge into an outer area, with both the dielectric and the first electrodes being permeable for said radiation.

DE 195 32 105 C2 discloses a method for treatment of three-dimensional workpieces with a direct barrier discharge which is induced by AC voltage, in which the workpiece surface to be treated is arranged opposite a first electrode, which is used for the AC-voltage-induced barrier discharge, on the workpiece side, has an electrically insulating barrier, in which the workpiece is used as the second electrode that is used for the AC-voltage induced barrier discharge, and in which at least the barrier is used with a surface which simulates the workpiece surface to be treated, with the barrier which simulates the surface being produced by casting, in particular by means of a thermoplastic. In this case, an appropriate casting process must be carried out in advance for each new topology, and this is associated with a relatively high level of time, material and therefore cost.

The problem on which the invention is based is therefore to provide an apparatus of this generic type which ensures uniform treatment via a plasma which is produced, for widely differing topologies, in particular for surfaces of the materials to be treated, in particular of skin.

According to the invention, this problem is solved by an apparatus as claimed in claim 1, by uses as claimed in claims 18 to 20, and by a method as claimed in claim 21.

The apparatus according to the invention is characterized in that this apparatus has a flexible active surface which is directly adjacent to the plasma during the treatment.

According to the invention, the term active surface means a surface of the apparatus which is directly adjacent to the plasma during the treatment—that is to say when the plasma exists—and, by virtue of the general material characteristics, the material has a dielectric constant which is not equal to zero, thus forming a dielectric barrier for the gas discharge, and therefore having a corresponding effect. The dielectric itself is solid in the aggregate state and can, but need not be, coated with one or more materials, which, for the first time allows a certain amount of flexibility, for example, when, although the dielectric is solid, it is, however, in the form of powder and this powder is applied to or introduced into a material similar to rubber, which has visco-elastic characteristics and can be appropriately shaped.

According to the invention, it should be understood that the active surface or the surface of the dielectric has a reversibly deformable shape. This allows a mechanical matching capability to the local circumstances while at the same time providing the effect of forming a dielectric barrier for a corresponding gas discharge. It is, of course, also feasible for solid granular or powder dielectric to be located/arranged on a flexible substrate.

As already mentioned above, it is, however, also feasible—effectively diametrically opposite thereto—to provide an intrinsically rigid—in the sense of not being flexible—existing solid dielectric with a coating which as such is flexible and/or for the first time allows or improves the flexibility as such, thus providing a flexible active surface for the purposes according to the invention, with respect to the effect of the solid dielectric and its configuration.

The fundamental principle of the apparatus according to the invention is based on an object to be treated being subjected to a plasma which is produced by means of an electrode and an opposing electrode, with a dielectric being arranged between the object to be treated and the electrode such that a plasma is produced by means of a dielectric-barrier gas discharge, and this plasma is then applied to the object to be treated.

This excitation principle results in a cold gas discharge (plasma) being formed between the electrode and the treatment area. This makes it possible to treat surfaces and/or cavities at a short distance away (0.1-50 mm), that is to say without making contact, and/or resting thereon in a locally highly confined area and/or by arranging a plurality of flexible electrodes in a row or a fabric-like structure, even over a large area with a different topology. The specific characteristics of the plasma result not only in use for treatment, activation, functionalization and disinfection of the corresponding surfaces and/or cavities, but also in fields of use in the medical area, in particular for application to skin or else for internal applications.

The effects which can be used in this case comprise, for example, low-dose IN irradiation in the useful UV-A and UV-B wavelength band, and the reactive gas species in the gas discharge (plasma). The method therefore combines a plurality of effective effects, thus resulting in a reduction in irritation, a conveyance of microcirculation, an immuno-modulatory effect and a germicidal and fungicidal effect, which is in turn highly useful for an application for at least some shoe inserts. At the same time, the apparatus can also be used for scanning of surfaces and/or cavities, in particular of skin, since this allows the treatment of skin diseases with accompanying intensive irritation, or else the treatment of chronic lack of wound healing on the basis of microcirculation disturbances.

The apparatus according to the invention uses voltages in the range from 100 to 100,000 volts. The applied voltage (see FIG. 13) may be sinusoidal (a), pulsed (b1, b2, c1, c2, d1, d2) (unipolar or bipolar), may be in the form of a radio-frequency pulse (e), or may be in the form of a DC voltage (f). Combinations of different voltage forms can also be used. The electrode may be composed of electrically highly conductive materials, with the opposing electrode being composed of the same material and/or with the object to be treated forming the opposing electrode. Normally, the solid dielectrics are composed of glasses, ceramics or plastics.

The AC voltage frequency is normally 1 Hertz to 100 MHz. The plasma treatment application times are governed by the field of use and may extend from a few milliseconds through several minutes to several hours.

One essential feature according to the invention is, inter alia, the fact that the apparatus has a flexible active surface which is directly adjacent to the plasma during the treatment, particularly when the solid dielectric is equipped with a flexible surface, which, for example, can be provided by the dielectric being in the form of a granulate and/or a powder. However, this can also be achieved by the dielectric being arranged, for example as a fine powder, on and/or in a flexible hollow fiber, for example composed of glasses, ceramics or plastics, or by the dielectric itself being formed by a flexible hollow fiber. The hollow fiber may have an internal diameter of 0.5 μm to 2000 μm. The wall thicknesses are in the range from 10 μm to 2000 μm. The length of the hollow fibers and the effective active length associated with this may extend from a few millimeters to several meters. The electrical connection of a connection to the electrode or opposing electrode is ensured in particular and for example via a metallic contact at the end of the hollow fiber. By way of example and in particular, this is introduced into the hollow fiber such that it closes the hollow fiber, if necessary also in a gas-type manner, thus allowing a conductive connection. The hollow fiber, contact and connection are accommodated in a holder so as to allow a secure connection from the voltage supply to the contact.

Because of the flexibility of the active surface, the apparatus can even be applied in difficult situations such as cavities—for example in the case of open wounds—so as to ensure that the plasma has a uniform and homogeneous effect on the surface to be treated.

In this context, it is advantageous for the electrode to rest at least partially directly on the surface of the dielectric in order to build up as high a field strength as possible for the electrical field which is formed in the dielectric between electrode and the opposing electrode, and when the surface of a specific object/subject to be treated, for example in the case of skin, is located between the electrode and the opposing electrode.

In this context and as an alternative embodiment, it is advantageous if the electrode is separated at least partially by means of a spacer from the surface of the dielectric. If the spacer is in this way in the form of a conductive material, and therefore not a dielectric, with the spacer being designed to have an appropriate electrical conductivity between the electrical conductivity of the electrode (very highly conductive) and the electrical conductivity of the dielectric (poorly conductive to having an insulating effect), in order in this way to homogenize the electrical field vectors, this leads to the plasma propagating better and more uniformly over an area.

In this context, it is advantageous for the electrode to rest at least partially on the dielectric, as a coating, since this results in a highly flexible embodiment, particularly when the dielectric is in the form of a flexible hollow conductor.

However, it is also feasible for the electrode to be formed from solid material, as a result of which, if the dielectric is in the form of a flexible hollow fiber, the electrode is arranged, as solid material, securely in the flexible hollow fiber.

It is also advantageous for the electrode to be in the form of a granulate and/or a powder, in order in this way to ensure the flexibility (for example capability to bend) of at least a part of the apparatus.

However, it is also feasible and advantageous if, in the operating state, the electrode is an ionized gas, thus resulting in a particularly high degree of flexibility (inter alia capability to bend) of the fiber with an appropriate configuration of the dielectric on and/or in a flexible hollow fiber, or as the hollow fiber itself, since there is no core material as a solid material; a corresponding situation also applies to the configuration of the electrode as an electrically conductive fluid, for example and in particular as an aqueous saline solution.

For the application of the plasma to a surface, it is particularly advantageous for the apparatus according to the invention to have an opposing electrode since this allows the application and guidance of the plasma to be controlled better, in contrast to embodiments in which the object to be treated effectively acts as the opposing electrode.

Furthermore, it is advantageous for the apparatus according to the invention to have a gas extraction device, which, in particular is flexible, and/or a gas supply device, which in particular is flexible, in order in this way to allow the plasma that is produced to be controlled specifically by means of the gas discharge, in order, for example, to remove any undesirable oxygen radicals or nitrogen oxides as quickly as possible, and in order to specifically supply gases in order, for example, to cool the treatment area and/or to deliberately cause reactions on the surface and/or in cavities, and/or to stabilize the plasma. The term “flexible” means the capability to align and/or place the corresponding device in order to satisfy different topical requirements. By way of example, the gas supply device and gas extraction device may in this case be formed essentially by flexible tubes.

By way of example, the gas extraction device and/or the gas supply device may also be in the form of flexible hollow fibers, since this is particularly advantageous for providing and/or improving the flexibility of the overall system.

Finally, it is advantageous if at least the one hollow fiber intrinsically or with at least one other supporting element, for example in the form of a fiber, forms a fabric-like element, for example in the form of a nonwoven, since this nevertheless allows a relatively large surface to be treated to be treated uniformly, despite having a different topology. A fabric-like element such as this, for example in the form of a nonwoven, can be incorporated in fabrics and/or healing apparatuses such as bandages or prostheses.

The shape of the fabric may be configured as appropriate for its purpose. Possible shapes are, for example and in particular round or polygonal. The surface of a fabric-like element such as this may have an active area of 10 mm² up to 1 m², or more.

However, it is also feasible and advantageous for flexible electrodes, in particular a flexible gas supply and/or in particular flexible gas extraction, to be arranged such that a free plasma flame is formed. In this case, the flexible electrodes may have a dielectric barrier (shield) on one side or both sides. This embodiment makes it possible to apply a plasma to surfaces and/or cavities which are further away than the other stated embodiments (up to several cm). This embodiment works independently of the conductivity at the surface, and of its surface structure.

Furthermore, the flexible electrodes allow the plasma flame to be deflected by actuators and/or a position unit in the X and/or Y direction (chosen using any desired Cartesian system). This is particularly advantageous since this allows the plasma flame to be guided over the surface.

The invention will be explained and described further in the following text with reference to preferred exemplary embodiments, which are illustrated in the figures, in which:

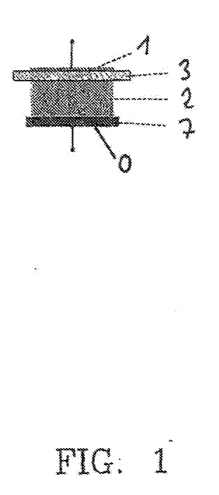

FIG. 1 shows a sketch of the functional principle of one embodiment from the prior art;

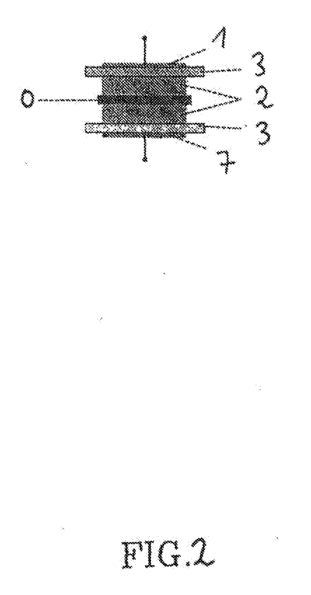

FIG. 2 shows a sketch of the functional principle of a further embodiment from the prior art;

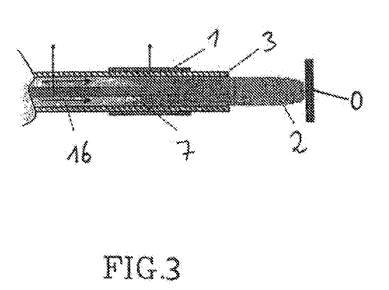

FIG. 3 shows a sketch of the functional principle of a third embodiment from the prior art;

FIG. 4 shows a sketch, in the form of a cross section, of a first embodiment according to the invention;

FIG. 5 shows a sketch, in the form of a cross section, of a second embodiment of the apparatus according to the invention;

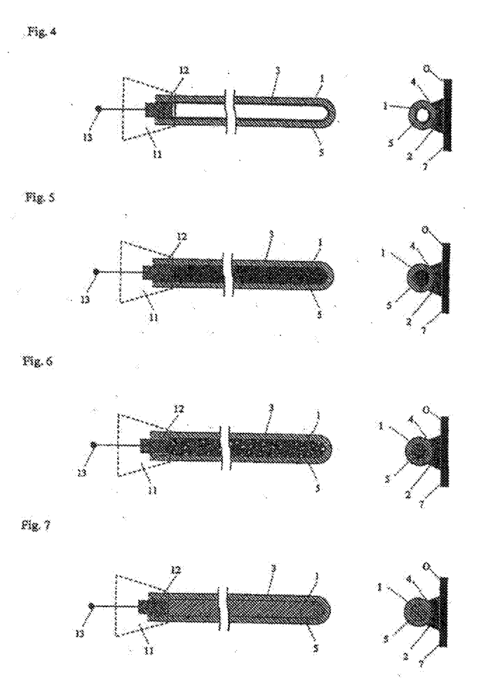

FIG. 6 shows a sketch, in the form of a cross section, of a third embodiment of the apparatus according to the invention;

FIG. 7 shows a sketch, in the form of a cross section, of a fourth embodiment of the apparatus according to the invention;

FIG. 8 shows a sketch, in the form of a cross section, of a fifth embodiment of the apparatus according to the invention;

FIG. 9 shows a sketch, in the form of a cross section, of a sixth embodiment of the apparatus according to the invention;

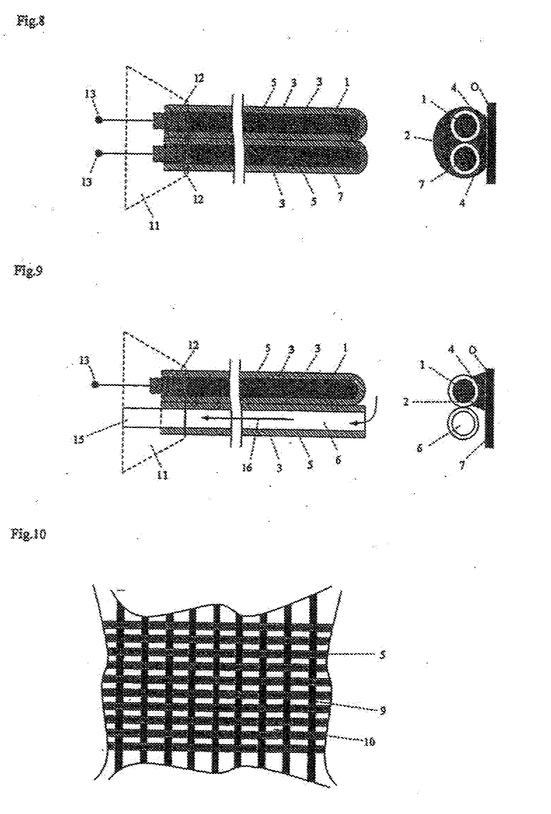

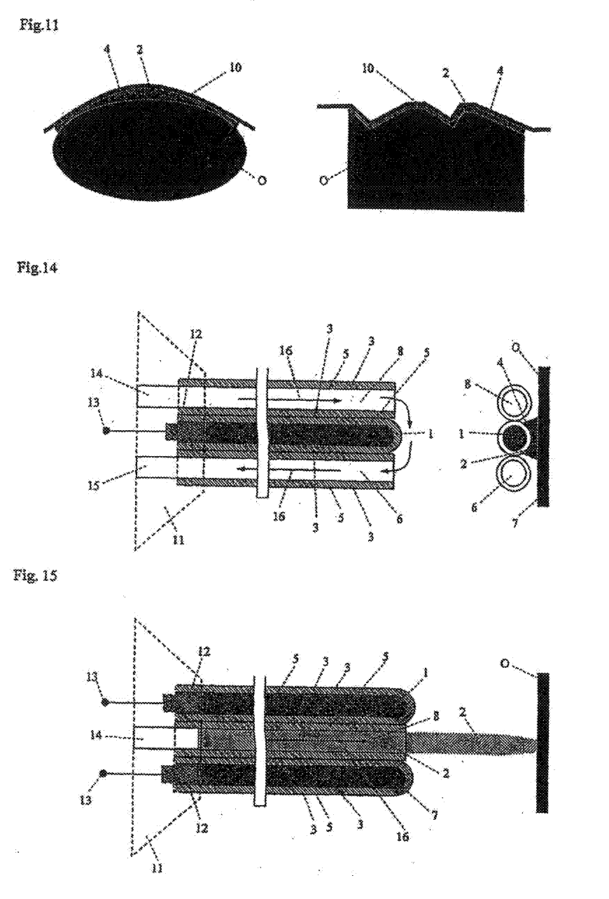

FIG. 10 shows a sketch, in the form of a cross section, of a seventh embodiment of the apparatus according to the invention;

FIG. 11 shows a sketch, in the form of a cross section, of a medical application;

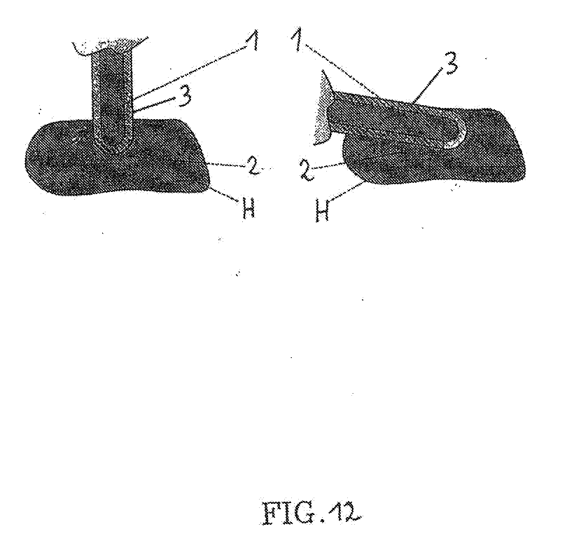

FIG. 12 shows a functional sketch of a conventional application of an apparatus according to the invention;

FIG. 13 shows a sketch of various voltage forms which can be applied to the electrode;

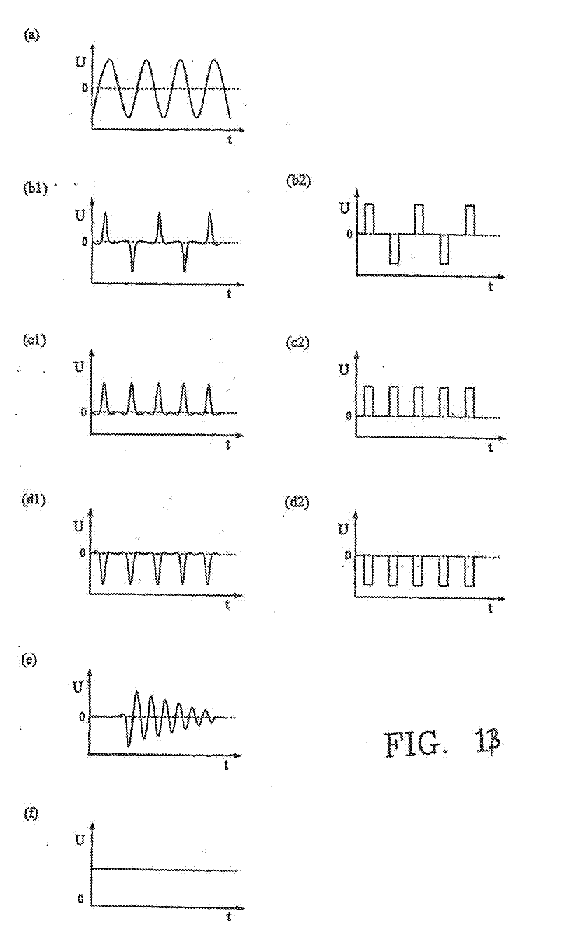

FIGS. 14-17 show sketches, in the form of cross sections, of eighth to eleventh embodiments.

FIG. 1 shows the functional layout of the apparatus according to the invention—as known from the prior art—in which an electrode (1) and the object O to be examined (conductively) acting as an opposing electrode 7 produce an electrical field when an AC voltage of several thousand volts and at frequencies up to the megahertz range is applied, in which air is converted by a corresponding gas discharge to a plasma 2 between the electrodes, as a result of which the object to be treated, as the opposing electrode 7, is treated directly topically by the plasma.

The principle (prior art) illustrated in FIG. 2 differs from that disclosed in FIG. 1 only in that the object to be treated is arranged between an electrode 1 and an opposing electrode 7, and is therefore located centrally in the plasma that is produced.

As can be seen from FIG. 3 (prior art), a corresponding plasma beam 2 is produced via a gas discharge, via a tubular supply of a gas to be ionized, by means of an electrode 1 and an opposing electrode 7, and this plasma beam is aimed directly at an object to be treated.

Fundamentally, in the case of the principles illustrated in FIGS. 1 and 2, an appropriate solid dielectric is located between the electrode and the object to be treated, with an appropriate solid dielectric 3 furthermore also being provided in FIG. 2, between the opposing electrode 7 and the object to be treated.

The following figures explain examples of various embodiments according to the invention.

In FIG. 4, the dielectric material is composed of glass, ceramic or plastic and is in the form of a flexible hollow fiber 5, with the inner wall of the hollow fiber 5 being coated with an electrically conductive material such as metals, doped semiconductors or conductive metal-oxide layers (ITO) (indium-tin oxide), with the coating acting as the electrode 1. In a configuration such as this, the object to be treated in general acts as the opposing electrode when only one hollow fiber 1 is used.

The embodiment shown in FIG. 5 differs from that shown in FIG. 4 only in that the electrode 1 is formed from solid material, and is composed of conductive materials such as metals and/or metal alloys or the like.

The embodiment shown in FIG. 6 differs from those shown in FIGS. 4 and 5 in that the electrode 1 is in the form of a powder, composed of conductive materials such as metals and/or metal alloys or the like.

The embodiment shown in FIG. 7 differs from the previous embodiments in that the electrode is in the form of a ionized gas, for example noble gases or other inert gases, or gas mixtures thereof, or is composed of other gases which can be ionized, with the ionized gas being produced, for example, in that the gas is ionized (plasma) by the application of a high voltage that is greater than the breakdown voltage. The ionized gas is now electrically conductive and can therefore be used as an electrode.

The embodiment shown in FIG. 8 differs from those shown in FIGS. 4, 5, 6 and 7 in that two corresponding hollow fibers 5 composed of dielectric material and each having solid-material electrodes are arranged adjacent to one another in the longitudinal direction, as a result of which the upper electrode acts as the electrode 1 and the lower electrode acts as the opposing electrode 5 when an appropriate voltage is applied, in such a way that the geometric arrangement of these two hollow fibers results in a specific plasma geometry, in which case, furthermore a plurality of hollow fibers are also feasible in order to produce a corresponding plasma geometry.

FIG. 9 differs from the embodiments shown in FIGS. 4, 5, 6 and 7 in that an appropriate extraction device 6 is arranged adjacent to the hollow fiber 5 in the longitudinal direction such that any undesirable components, for example oxygen radicals that are produced, are quickly removed from the object to be treated, for example in order not to irritate sensitive skin particles.

FIG. 14 differs from the embodiments shown in FIGS. 4, 5, 6 and 7 in that an appropriate, flexible gas extraction device 6 and a flexible gas supply device 8 are arranged adjacent to the hollow fiber 5 in the longitudinal direction, such that any undesirable components, for example oxygen radicals that are produced, are quickly removed from the object to be treated, or else to specifically supply gases in order, for example, to cool the treatment area and/or to deliberately cause reactions.

As can be seen from FIG. 10, a plurality of hollow fibers 5 composed of dielectric material, or having a dielectric coating composed of glass, ceramic or plastic, and provided with electrodes, for example in the form of an inner coating (see the embodiment in FIG. 4) in conjunction with further supporting elements 9 in the form of fibers form a fabric-like element 10, thus allowing appropriately adequate and matched shaping, and therefore application, even in the case of difficult topologies (see FIG. 11).

Finally, FIG. 12 shows a sketch of a conventional application of the apparatus according to the invention with respect to a part of a skin area H (in this case, the skin H is the object O to be treated), acting as an opposing electrode and object.

FIGS. 15 to 17 show different embodiments, which differ from the previous embodiments in that the electrode or electrodes and/or the gas supply device are/is arranged such that a free plasma flame is formed. The free plasma flame of the plasma 2 emerging from the flexible apparatus can be used for direct topical application.

In the embodiment illustrated in FIG. 15, the plasma is provided, with a dielectric barrier, by means of appropriate solid dielectrics 3, in order to prevent direct contact, with respect to the electrode 1 and the opposing electrode 7.

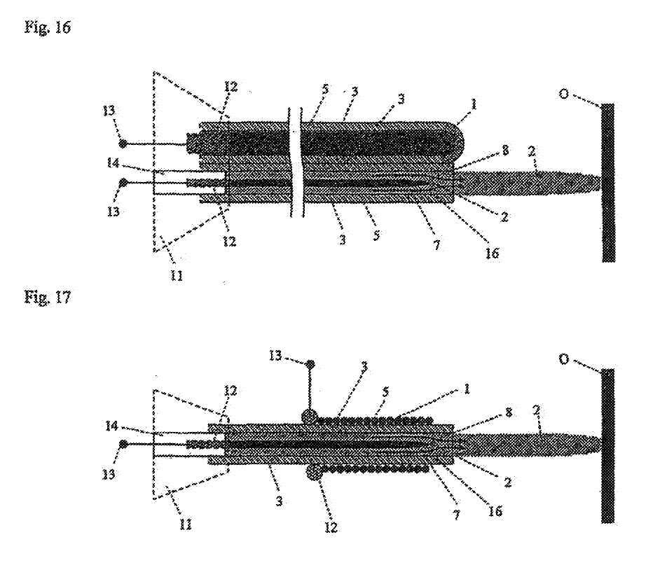

In the embodiments in FIGS. 16 and 17, only one simple dielectric barrier is provided, such that the plasma cannot make direct contact with the electrode 1 through the solid dielectric 3, but makes direct contact with the opposing electrode 7, since this is located in the plasma itself and, for example, is in the form of an electrically conductive flexible wire.

The embodiment in FIG. 17 differs essentially from that in FIG. 16 in that the electrode 1 is arranged in a spiral shape as an outer electrode around the solid dielectric (in this case: hollow-fiber material) in order to provide and/or to assist a certain amount of mechanical flexibility. It is, of course, also feasible for the embodiments shown in FIGS. 15 to 17 to be equipped with a gas extraction device as shown in FIG. 14.

### LIST OF REFERENCE SYMBOLS

O—Object
H—Skin
1—Electrode
2—Plasma
3—Solid dielectric
4—Active surface
5—Hollow fiber
6—Gas extraction device
7—Opposing electrode
8—Gas supply device
9—Supporting element
10—Fabric-like element
11—Holder
12—Contact
13—Connection, electrical
14—Gas inlet
15—Gas outlet
16—Gas flow

1. An apparatus for treatment of surfaces with a plasma (2) which is produced by means of an electrode (1) over a solid dielectric (3) by a dielectric-barrier gas discharge, wherein the apparatus has an active surface (4) which is directly adjacent to the plasma (2) during the treatment, characterized in that the active surface has a reversibly variable shape.

2. The apparatus as claimed in claim 1, characterized in that the dielectric (3) has a surface (4) with a reversibly deformable shape.

3. The apparatus as claimed in claim 1, characterized in that the dielectric (3) is arranged on and/or in a flexible hollow fiber (5).

4. The apparatus as claimed in claim 1, characterized in that the dielectric (3) is a flexible hollow fiber (5).

5. The apparatus as claimed in claim 1 4, characterized in that the dielectric (3) is in the form of a granulate and/or a powder.

6. The apparatus as claimed in claim 1, characterized in that the electrode (1) rests at least partially directly on the active surface of the dielectric (3).

7. The apparatus as claimed in claim 1, characterized in that the electrode (1) is separated at least partially by means of a spacer from the active surface of the dielectric.

8. The apparatus as claimed in claim 6, characterized in that the electrode (1) rests at least partially on the dielectric (3), as a coating.

9. The apparatus as claimed in claim 6, characterized in that the electrode (1) is formed from solid material.

10. The apparatus as claimed in claim 6, characterized in that the electrode (1) is in the form of a granulate and/or a powder.

11. The apparatus as claimed in claim 6, characterized in that the electrode (1) is in the form of an electrical conductive fluid.

12. The apparatus as claimed in claim 6, characterized in that, in the operating state, the electrode (1) is an ionized gas.

13. The apparatus as claimed in claim 1, characterized in that the apparatus has an opposing electrode (7).

14. The apparatus as claimed in claim 1, characterized in that the apparatus has a gas extraction device (6) and/or a gas supply device (8)

15. The apparatus as claimed in claim 14, characterized in that the gas extraction device (6) and/or the gas supply device (8) are/is flexible.

16. The apparatus as claimed in claim 3, characterized in that at least the one hollow fiber (5) intrinsically or with at least one other supporting element (9) forms a fabric-like element (10)

17. The apparatus as claimed in claim 1, characterized in that the electrode and/or the gas supply device and/or the gas extraction device are/is arranged such that a free plasma flame is formed.

18. The use of an apparatus as claimed in claim 1, for treatment, activation and functionalization of surfaces and/or cavities, in particular of skin.

19. The use of an apparatus as claimed in claim 1, for disinfection and/or cleaning of surfaces and/or cavities, in particular of skin.

20. The use of an apparatus as claimed in claim 1, at least as part of shoe inserts and/or textile inserts and/or medical bandaging and/or a healing and/or supporting apparatus.

21. A method for treatment and/or disinfection of surfaces and/or cavities, in particular of skin, having a plasma produced by a dielectric-barrier gas discharge, characterized by the use of an apparatus as claimed in claim 1.
